(19) United States
(12) Patent Application Publication
(10) Pub. No.: US 2003/0120181 A1
(43) Pub. Date: Jun. 26, 2003

(54) Work-hardened pseudoelastic guide wires

(75) Inventors: Mark Toma, Sherman, CT (US); John Tommessilli, New Haven, CT (US); Scott Carpenter, Fremont, CA (US); Jesse Perez, Newark, CA (US)

Correspondence Address: PERKINS, SMITH & COHEN LLP, ONE BEACON STREET, 30TH FLOOR, BOSTON, MA, 02108, US

(73) Assignee: Memry Corporation

(21) Appl. No.: 10/287,140

(22) Filed: Nov. 4, 2002

Related U.S. Application Data
(60) Provisional application No. 60/338,719, filed on Nov. 5, 2001.

(51) Int. Cl. A61B 5/00

(52) U.S. Cl. 600/585

(57) ABSTRACT

The present invention provides a medical guide wire and a method of making same in which, an elongated solid core wire is made of NiTi alloy with a Ni content of about between 55.0 and 56.5 wt % and a reverse martensitic transformation start temperature (As) in the fully annealed state of not more than 55° C. The wire has been thermomechanically processed to exhibit a work-hardened pseudoelasticity. After the last full annealing to regain workability, the wire is cold drawn with a significant amount of cold reduction of greater than 35%, but preferably greater than 38% The entire guide wire is subjected to the same heat treatment. The wire is formed into an elongated solid core. The heating step includes passing the wire through a tube furnace at substantially 280° C. to 370° C. The entire guide wire is subjected to the same heat treating step. The guide wire has centerless grinding performed at an appropriate stage to provide a taper section and a distal section. There may be a coil attached around the distal section of the guide wire and which is made of a deformable material so that it may be deformed to a different radius or angle. Later, an outer jacket is provided which surrounds the core.

### CROSS REFERENCE TO RELATED APPLICATIONS AND PATENTS

The present application has the benefit of the filing date of U.S. Provisional Application No. 60/338,719 filed Nov. 5, 2001, the content of which is incorporated herein by reference.

### FIELD OF THE INVENTION

The present invention relates generally to medical guide wires, and, more particularly, to guide wires for navigating the passages of blood vessels, trachea, gastrointestinal tracts, and other channels or cavities of a human body.

### BACKGROUND OF THE INVENTION

With the recent progress of minimally invasive surgical techniques, medical devices have been designed to function through catheters that are guided to the surgical site through the network of blood vessels, trachea, gastrointestinal tracts, or other cavities of human anatomy. During an intraluminal procedure, the physician first introduces a guide wire through a punctured hole into the vessel. The physician then manipulates the guide wire by pushing and torqueing while observing the advancement through the vessels on a fluoroscope to access the targeted site. Once a desired location is reached, catheters or devices may then be delivered over the guide wire to a specific site of interest for either diagnosis or therapy. The guide wire is then removed.

In order for a physician to easily navigate through, very often-torturous, networks of these channels without traumatizing the vessel wall, an ideal guide wire must have a balance of flexibility and an ability to transfer push and torque through the length of the wire. This balance provides good steerability and allows the physician to insert the wire percutaneously and then advance the wire through the tortuous passages and bifurcated branches to a target site. The distal portion of the wire must be flexible to a point that it is atraumatic to the vessel wall, but the body portion must be stiff enough to act as a guide rail for other devices such as angiographic catheters, balloon catheters, and stent delivery systems, to be advanced over the wire to the target site. To achieve this balance, guide wires of a general metallic material such as stainless steel has a common construction that the cross section near the distal end is gradually reduced toward the tip. A coiled spring may be attached to the distal portion to further enhance the flexibility and to reduce the risk of traumatizing the vessel. Tips may be straight, angled or J-shaped to help navigating and accessing branching of tortuous vessels. The cross section of the body portion is maintained over the majority of the length with a smooth outside diameter whereby the portion is comparatively rigid to transfer the push and torque for manipulation and to support in guiding the delivery of catheter, stent or other intraluminal devices. Yet, the body portion must also possess sufficient flexibility in order for the wire to easily conform to the vessel anatomy.

During the advancement of the guide wire, the physician may reposition the wire several times to reach a target site, very often by navigating the wire through bend regions of tight radii. In addition, the manipulation of the ancillary devices may significantly deform the wire well over the elastic limit leading to plastic deformations or kinking of the wire. Kinks at any portion of the guide wire interfere with the navigation and make it more difficult to advance the auxiliary devices during subsequent delivery. An ideal guide wire, therefore, must also be reasonably kink resistant.

Superelastic NiTi guide wire offers an excellent combination of flexibility, pushability, torqueability and kink-resistance. The benefits of superelastic NiTi guide wire over stainless steel guide wire have been discussed in detail in a publication by Fernald et al., “NiTi: The Material Of Choice For High Performance Guide Wires”, Proceedings of the First International Conference on Shape Memory and Superelastic Technologies, Pacific Grove, Calif., p.341, 1994. NiTi alloys belong to a class of shape memory alloy which exhibits thermoelastic martensitic crystalline phase transformation. The term “martensite” refers to the crystalline phase present at low temperatures while the phase that exists at elevated temperatures is referred to as “austenite”. Thermoelastic martensitic transformation occurs as a reversible and diffusionless crystalline phase change over a small temperature span. During the transformation on cooling, the high temperature austenitic phase changes its crystalline structure through a diffusionless shear process adopting a less symmetrical structure of martensite, and, on heating, the reverse transformation occurs with a small thermal hysteresis. The starting temperature of the cooling transformation is referred to as the Mₛ temperature and the finishing temperature, M_{f}. The starting and finishing temperatures of the reverse transformation on heating are referred to as Aₛ and A_{f}, respectively.

For certain NiTi alloys, a similar crystalline phase change precedes the martensitic transformation resulting in an intermediate phase having a rhombohedral crystalline structure which is referred to as “R-phase”. In case of this two-stage transformation, the starting and finishing temperatures for the austenite transforming into R-phase on cooling are referred to as Rₛ and R_{f} temperatures, respectively. The starting and the finishing temperatures for the reverse transformation from martensite to R-phase are referred to as Rₛ′ and R_{f}′, respectively. The definition of transformation temperatures for NiTi shape memory alloys has been standardized in ASTM F2005, “Standard Terminology for Nickel-Titanium Shape Memory Alloys”.

Alloys undergoing thermoelastic martensitic transformation may exhibit “shape memory effect” and “pseudoelasticity”. Materials exhibiting shape memory effect can be deformed in their martensitic phase and upon heating recover their original shapes. These materials can also be deformed in their austenitic phase above the A_{f} temperature through stress-induced martensitic transformation and recover their original shapes upon the release of stress. Both the loading and unloading occur at relatively constant stress exemplified by plateaus in the stress-strain curve. This strain recovery referred to as “pseudoelasticity” is associated with the reversion of stress-induced martensite back to austenite. “Superelasticity” is often used alternatively for “pseudoelasticity” and both have been used to describe this transformation-induced nonlinear elasticity. A general review on shape memory alloys can be found in “Shape Memory Alloys” by Hodgson et al in volume 2 of Metals Handbook, 10^{th} edition, p.897, 1990.

A typical stress-strain curve of pseudoelastic NiTi alloys exhibits flat plateaus on both loading and unloading sections related to the stress-induced martensitic and the reverse transformation, respectively, as illustrated in FIG. 1. Pseudoelastic NiTi guide wires are typically manufactured by die drawing to proper diameters followed by strand annealing under tension at a temperature between 400° C. and 600° C. NiTi wires in the cold drawn condition exhibit linear superelasticity as shown in FIG. 2. Strain as high as 3% can be recovered. The cold drawn wires are subsequently heat treated by passing through a tubular furnace under tension. For the purpose of clarity, “pseudoelasticity” will be used herein to describe elasticity related to stress-induced transformation where plateaus are present in the stress-strain curve while “superelasticity” will be used to describe linear elasticity of the cold-worked NiTi material.

A guide wire made of a pseudoelastic shape memory alloy such as NiTi has been disclosed in U.S. Pat. No. 4,925,445. A pseudoelastic alloy is used which has a temperature at which transformation to austenite is complete at most about 10° C. At body temperature, the alloy exhibits stress-induced pseudoeaslticity having well-defined loading plateau and unloading plateau characterized by deformation at relatively constant stresses. Pseudoelastic NiTi guide wires have the advantages of being highly flexible and kink-resistance but the pseudoelasticity makes them difficult to form the distal portion to any desirable shape. In addition, the wires may have insufficient body stiffness and therefore a less than ideal steerability.

U.S. Pat. No. 5,069,226 discloses a NiTiFe guide wire having a balanced pseudoelasticity and plasticity such that the distal portion is readily formable into a desirable shape. Also disclosed in the patent is a NiTi guide wire where the distal tip is heat-treated at 700° C. to gain plasticity while the remainder portion is in either cold-worked condition or heat-treated at a temperature less than 400° C. whereby the portion exhibits elasticity but no pseudoelasticity NiTi guide wires having distinct elasticity between the distal and the remainder portions require either joining or discrete heat treatment of multiple passes and are thus more difficult and costly to manufacture.

U.S. Pat. No. 5,120,308 describes a catheter with high tactile guide wire where the guide wire is a NiTi wire exhibiting either pseudoelasticity or linear superelasticity.

Another NiTi guide wire is described in U.S. Pat. No. 5,238,004 wherein at least the distal portion comprises a linear elastic NiTi alloy that is in a precursor state of a superelastic alloy. NiTi alloy in this state exhibits martensitic structure and linear elasticity without any transformation induced plateau of pseudoelasticity. A linear superelastic guide wire has a higher stiffness and better torque transfer characteristic than does a superelastic guide wire. However, straightness is not easily obtainable by mechanical straightening.

WO00/27462 discloses methods of mechanical straightening of linear elastic NiTi guide wire under the assist of predetermined twisting shear strain, tension and temperature. Although both discrete and continuous methods were disclosed, applying twisting shear strain onto a continuous spool of wire is difficult to control, imposing a significant limitation for the continuous manufacturing process.

### BRIEF SUMMARY OF THE INVENTION

The present invention provides a medical guide wire and a method of making same in which, an elongated solid core wire is made of NiTi alloy with a Ni content of about between 55.0 and 56.5 wt % and a reverse martensitic transformation start temperature (As) in the fully annealed state of not more than 55° C. The wire has been thermomechanically processed to exhibit a work-hardened pseudoelasticity. After the last full annealing to regain workability, the wire is cold drawn with a significant amount of cold reduction of greater than 35%, but preferably greater than 38% The entire guide wire is subjected to the same heat treatment. The wire is formed into an elongated solid core. The heating step includes passing the wire through a tube furnace at substantially 280° C. to 370° C. The entire guide wire is subjected to the same heat treating step.

The guide wire has centerless grinding performed at an appropriate stage to provide a taper section and a distal section of smaller diameter than the core. There may be a coil attached around the distal section of the guide wire and which is made of a deformable material so that it may be deformed to a different radius or angle. Later, an outer jacket is provided which surrounds the core.

Other objects, features and advantages will be apparent from the following detailed description of preferred embodiments taken in conjunction with the accompanying drawings in which:

### BRIEF DESCRIPTION OF THE DRAWINGS

FIG. 1. Stress-strain curve of a pseudoelastic NiTi wire.

FIG. 2. Stress-strain curve of a cold-worked linearly superelastic NiTi wire.

FIG. 3. Sectional view of a guide wire of present invention.

FIG. 4. Stress-strain curve of a work-hardened pseudoelastic NiTi wire.

FIG. 5. Section view of a guide wire of present invention including a coil element attached to the distal section.

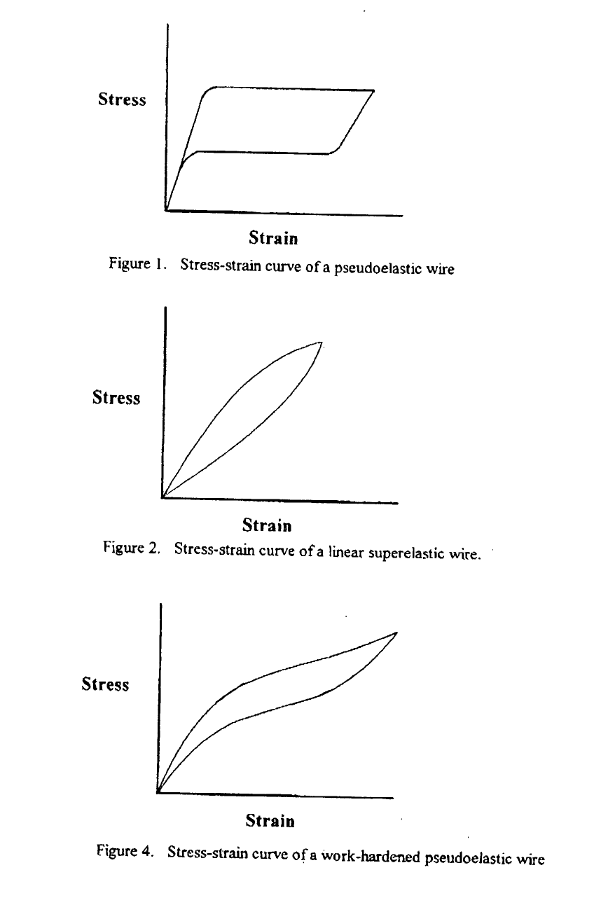

### DESCRIPTION OF THE EMBODIMENTS

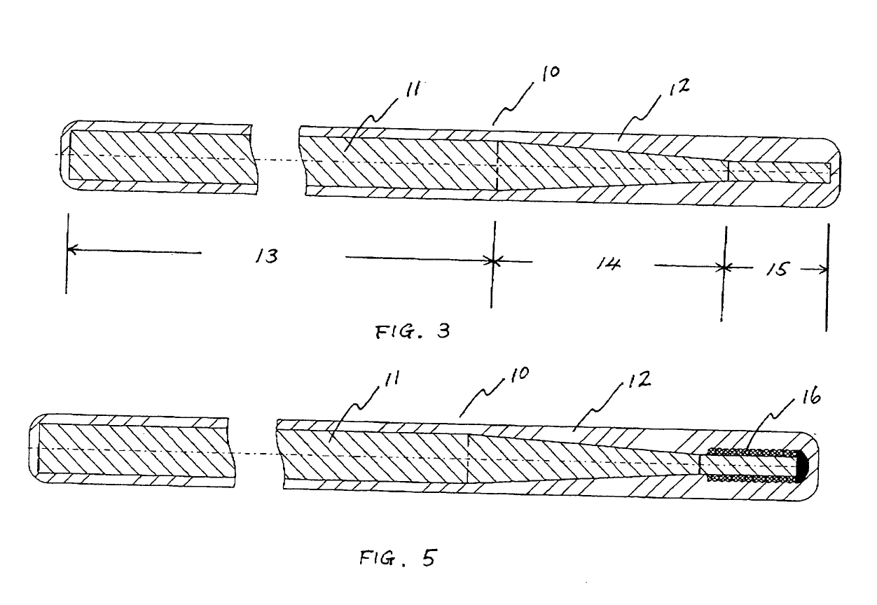

FIG. 3 illustrates a guide wire 10 of an embodiment within the present invention. The article comprises an elongated solid core wire 11 and an outer jacket 12. The elongated solid core wire 11 includes a proximal section 13 of a constant diameter, a tapered section 14 and a distal section 15 of a smaller constant diameter than the proximal section. The core wire is made of a NiTi alloy where the Ni content is in the range of 55.0 to 56.5 weight percent and a reverse martensitic transformation start temperature (As) in the fully annealed state less than or equal to 55C. The entire wire, including the distal section, is thermomechanically processed to exhibit a work-hardened pseudoelasticity at a temperature about 37C, characterized by slanted loading and unloading plateau in stress-strain curves of deformation, as illustrated in FIG. 4.

The NiTi core wire is formed by repetitive drawn and annealing to a final usable diameter. After the last full annealing to regain workability, the wire is cold drawn with a significant amount of cold reduction greater than 35% reduction in cross-section area, but preferably greater than 38%. The wire is then heat treated to impart the final properties of work-hardened pseudoelasticity. The preferred heat-treatment process involves passing the wire under tension through a tube furnace heated to a temperature of 280-370° Celsius, at a run rate that the wire is heat treated at the temperature for a duration in the range of approximately 10-40 seconds. Preferred tension is in the range of approximately 8,000-20,000 pounds per square inches. Wires after this heat treatment at proper conditions exhibit work-hardened pseudoelastic stress-strain characteristic as depicted in FIG. 4. It is understood that fine structures of metallurgical recovery and early stage of recrystallization cause continuously rising plateau stresses during the stress-induced martensitic transformation. A work-hardened pseudoelastic NiTi guidewire of the present invention exhibits a greater stiffness, and hence a better torque-transfer characteristic, than a pseudoelastic NiTi guide wire. It can be deformed to a higher degree of strain without imparting a significant plastic deformation, and hence better kink resistance, than a linear elastic NiTi guide wire. The characteristics detailed above can be more easily achieved with good straightness than of linear superelastic core wires through a combination of manufacturing process involving drawing and heat-treating and post process grinding.

### EXAMPLE 1

The NiTi alloys useful for present invention are normally melted and cast using vacuum induction or vacuum arc melting process. The ingots are then forged, rolled and drawn into wires. In one example, the aforementioned core wire 11 of 0.028 inch in diameter was formed of a NiTi alloy having a nominal composition of 55.0 weight percent Ni and an austenite transformation start (As) temperature of 45 degree C. in the fully annealed state. The wire after being cold drawn with a 50 percent reduction in cross-section area was heat treated by passing the wire through a tube furnace at 325 degree C. under a longitudinal tension of 16,000 pounds per square inch (psi), and at a speed that corresponds to a duration of 36 seconds. The core wire after being formed of such a process exhibited work-hardened pseudoelasticity and a tensile strength of 122,000 psi at 4% strain. After being tensile tested to 6% longitudinal strain, the residual strain after unloading is about 0.16%.

### EXAMPLE 2

In another example, the core wire 11 of 0.023 inch in diameter was formed of a NiTi alloy having a nominal composition of 55.8 weight percent Ni and an austenite transformation start (As) temperature of −15 degree C. in the fully annealed state. The wire was cold drawn to the finish diameter with a 40 percent reduction in cross-section area and subsequently heat treated through a tube furnace at 350 degree C. at a speed that yielded heat treatment duration of 19 seconds. A longitudinal tension of 19,000 psi was applied to maintain straightness during the heat treatment. The heat-treated wire exhibited work-hardened pseudoelasticity, a tensile strength of 83,800 psi at 4% strain and nil permanent deformation after testing to 4% longitudinal strain.

### EXAMPLE 3

In another example, the core wire 11 of 0.024 inch in diameter was made of a NiTi alloy having a nominal composition of 55.8 weight percent Ni and an austenite transformation start (As) temperature of −15 degree C. in the fully annealed state. The wire was cold drawn to the finish diameter with a 45% reduction in cross-section area and subsequently heat treated under a longitudinal tension of 17,700 psi through a tube furnace at 350 degree C. at a speed corresponding to heat treatment duration of 14 seconds. The heat-treated wire exhibited work hardened pseudoelasticity with a tensile strength of 104,000 psi at 4% strain and a permanent deformation of 0.02% after tensile testing to 4% deformation.

### EXAMPLE 4

In another example, the core wire 11 made of a NiTi alloy of nominally 55.8 weight percent Ni was drawn from a 0.030-inch diameter pseudoelastic wire to 0.024 inch in finish diameter with a 38% reduction in cross section area. The wire was then mechanically straightened and heat-treated by passing through a tube furnace at 370 degree C. under a longitudinal tension of 11,000 psi and at a speed corresponding to heat treatment duration of 12 seconds. The wire after the process exhibited work hardened pseudoelasticity with a tensile strength of 107,000 psi at 4% strain and a permanent deformation of 0.33% after testing to 4% deformation.

### EXAMPLE 5

In yet another example, the core wire 11 made of a NiTi alloy of nominally 55.8 weight percent Ni was drawn from a 0.030-inch diameter pseudoelastic wire to 0.024 inch in finish diameter with a 38% reduction in cross section area. The wire was then mechanically straightened and heat-treated by passing through a tube furnace at 360 degree C. under a longitudinal tension of 11,000 psi and at a speed corresponding to heat treatment duration of 12 seconds. The wire after the process exhibited work hardened pseudoelasticity with a tensile strength of 100,000 psi at 4% strain and a permanent deformation of 0.23% after testing to 4% deformation.

Referring to FIG. 5, a coil element 16 may be attached, for example, to the distal section of the core wire by solder joining method. The coil element may be formed of stainless steel or of noble materials, such as platinum, with good radiopacity so the position of the guide wire during the procedure can be easily monitored by radiography. Because the distal tip of a work-hardened pseudoelastic core wire is difficult to deform or shape, it is preferable that the coil element being stiffer than the distal section of the core wire so that the distal section with the attached coil element can be shaped into desirable curvatures.

After the core wire is heat-treated, cut to length, ground to have a flexible distal section, and attached with a distal coil element, the wire is jacketed with a polymer, and coated with a hydrophilic polymer. The polymer jacket 12 may be polyethylene, polyester, polyvinyl chloride, fluoride resin or any other synthetic resins or elastomers. The jacket 12 may also be made of polymer blended with powders or compounds of Ba, W, Bi, Pd or other radiopaque elements to enhance the visibility of guide wire under radioscopy during medical procedure. The total length of the wire and grind profile will vary depending upon the specific procedure and physician skill or preference. The polymer jacket is added to assist the tip from kinking or piercing tissue as well as to provide a smooth surface to advance the ancillary devices. The hydrophilic polymer provides a lubricious surface to help assist the advancement through highly tortuous vessels.

It will now be apparent to those skilled in the art that other embodiments, improvements, details, and uses can be made consistent with the letter and spirit of the foregoing disclosure and within the scope of this patent, which is limited only by the following claims, construed in accordance with the patent law, including the doctrine of equivalents.

1. A medical guide wire made of a NiTi alloy wherein at least a portion thereof is characterized by being stiffer and having better torqueability than a guide wire of a pseudoelastic NiTi alloy, but being more flexible than a guide wire of a linearly elastic NiTi guide wire, thereby providing a good combination of flexibility and kink resistance to allow the guide wire to navigate through the highly torturous passages such as blood vessels, trachea, gastrointestinal tracts, and other cavities of a human body.

2. A guide wire as defined in claim 1, which further exhibits a slanted plateau and a mechanical hysteresis during the loading and unloading sections of its stress-strain curve.

3. A guide wire as defined in claim 2 wherein said portion is made of a work-hardened pseudoelastic shape memory alloy, that has been cold worked and heat treated.

4. A medical guide wire, comprising:
an elongated solid core wire made of NiTi alloy with a Ni content of about between 55.0 and 56.5 wt % and a reverse martensitic transformation start temperature (As) in the fully annealed state of not more than 55° C., said wire having been thermomechanically processed to exhibit a work-hardened pseudoelasticity.

5. A guide wire as defined in claim 4 wherein the pseudoelasticity is exhibited at a temperature of about 37° C.

6. A guide wire as defined in claim 4 wherein after the last full annealing to regain workability, the wire having been cold drawn with a significant amount of cold reduction of greater than 35%.

7. A guide wire as defined in claim 6, the wire having been heat treated by passing through a tube furnace at 280° to 370° C.

8. A guide wire as defined in claim 4, further comprising a coil surrounding the distal section of the guide wire.

9. A guide wire as defined in claim 8, where said coil may be deformed to a different radius and/or angle.

10. A guide wire as defined in claim 7, the wire having been under a longitudinal tension of substantially 8,000 to 20,000 psi during the heat treatment.

11. A guide wire as defined in claim 4 wherein the original wire prior to processing is substantially 0.023 to 0.030 inch diameter.

12. A guide wire as defined in claim 7 the wire having been heat treated for approximately 10 to 40 seconds.

13. A guide wire as defined in claim 4, the wire exhibiting a tensile strength of substantially 83,300 to 122,000 psi at 4% strain.

14. A guide wire as defined in claim 13, the wire exhibiting a permanent deformation of 0 to 0.33% after tensile testing to 4-6% deformation.

15. A guide wire as defined in claim 4, further comprising an outer jacket surrounding said core.

16. A guide wire as defined in claim 7, the entire guide wire having been subjected to the same heat treatment.

17. A method of making a medical guide wire, comprising the steps of:
a. forming a wire of NiTi alloy with a Ni content of about between 55.0 and 56.5 wt %, which has a reverse martensitic transformation start temperature (As) in the fully annealed state of not more than about 55° C.;
b. fully annealing the wire to regain workability;
c. cold drawing the wire with a significant amount of cold reduction of greater than about 35% in cross-sectional area;
d. heat treating the wire to exhibit a work-hardened pseudoelasticity; and
e. forming the wire into an elongated solid core.

18. A method as defined in claim 17 wherein the heat treating step includes passing the wire through a tube furnace at 280° C. to 370° C.

19. A method as defined in claim 18, wherein the wire is under a longitudinal tension of substantially 8,000 to 20,000 psi during the heat treating step.

20. A method as defined in claims 17, wherein the original wire prior to processing is substantially 0.023 to 0.030 inches in diameter.

21. A method as defined in claim 18, wherein the heat treating is carried out for substantially 10 to 40 seconds.

22. A method as defined in claim 18 wherein the entire guide wire is subjected to the same heat treating step

23. A method as defined in claim 22, wherein the guide wire is subjected to center-less grinding to provide a taper section and a distal section of smaller diameter than the core.

24. The method as defined in claim 17 further comprising the step of placing a coil around the distal section of the guide wire, said coil being made of a deformable material so that it may be deformed to a different radius or angle.

25. The method as defined in claim 17 further comprising the step of providing an outer jacket which surrounds the core.
